(19) United States
(12) Patent Application Publication
(10) Pub. No.: US 2016/0327508 A1
(43) Pub. Date: Nov. 10, 2016

(54) Sensors For Carbon Dioxide And Other End Uses

(71) Applicant: Dioxide Materials, Inc., Boca Raton, FL (US)

(72) Inventors: Richard I. Masel, Boca Raton, FL (US); Brian Rosen, Wilmington, DE (US)

(21) Appl. No.: 15/148,690

(22) Filed: May 6, 2016

Related U.S. Application Data
(63) Continuation-in-part of application No. 13/530,058, filed on Jun. 21, 2012, now abandoned.
(60) Provisional application No. 61/499,225, filed on Jun. 21, 2011.

(51) Int. Cl. G01N 27/407; G01N 27/327; G01N 33/487; G01N 27/333

(52) U.S. Cl. CPC G01N 27/4075; G01N 27/4074; G01N 27/3335; G01N 27/3273; G01N 33/48707

(57) ABSTRACT

Electrochemical sensors measure an amount or concentration of CO₂, typically using catalysts that include at least one Catalytically Active Element and one Helper Catalyst. The catalysts can be used to increase the rate, modify the selectivity or lower the overpotential of chemical reactions. These catalysts are useful for a variety of chemical reactions including electrochemical conversion of CO₂. Chemical processes and devices employing the catalysts are also disclosed, including processes that produce CO, OH⁻, HCO⁻, H₂CO, (HCO₂)⁻, H₂CO₂, CH₃OH, CH₄, C₂H₄, CH₃CH₂OH, CH₃COO⁻, CH₃COOH, C₂H₆, O₂, H₂, (COOH)₂, and (COO⁻)₂.

### CROSS-REFERENCE TO RELATED APPLICATIONS

The present application is a continuation-in-part of U.S. patent application Ser. No. 13/530,058 filed on Jun. 21, 2012, entitled “Sensors For Carbon Dioxide And Other End Uses”. The '058 application was related to and claimed priority benefits from U.S. provisional patent application Ser. No. 61/499,225, filed on Jun. 21, 2011, entitled “Low Cost Carbon Dioxide Sensors”. The present application is also related to and claims priority benefits from the '225 provisional application. The '225 provisional application and the '058 non-provisional application are each hereby incorporated by reference in their entirety.

### STATEMENT OF GOVERNMENT INTEREST

This invention was made, at least in part, with U.S. government support under U.S. Department of Energy Grant DE-SC0004453. The government has certain rights in the invention.

With respect to the above-mentioned parent and predecessor applications, to the extent any amendments, characterizations or other assertions previously made in any such related patent applications or patents, including any parent, co-pending or continuing application with respect to any art, prior or otherwise, could be construed as a disclaimer of any subject matter supported by the disclosure of the present application, such disclaimer is hereby rescinded and retracted. Prior art previously considered in any related patent application(s) or patents(s), including any parent, co-pending or continuing application, should be reconsidered with respect to the subject matter being claimed in the present application.

### FIELD OF THE INVENTION

The present invention relates to electrochemical sensors, particularly those that sense carbon dioxide and other chemical substances in end uses such as control systems for heating, ventilation and air conditioning (HVAC).

### BACKGROUND OF THE INVENTION

Carbon dioxide (CO₂) sensors are useful (1) to monitor gaseous emissions, (2) as a sensor to adjust ventilation rates in buildings and thereby lower heating costs and improving air quality, and (3) as a patient monitor for medical uses, and for other applications in which the detection and monitoring of carbon dioxide is necessary or desirable. These types of applications are growing because of concerns about greenhouse gas emissions and rising energy costs.

Several different carbon dioxide sensor designs have been previously disclosed. Infrared or near infrared sensors are the most common, but they are limited by their high cost. Semiconductor devices have been used, but they have limited selectivity. Solid oxide electrolyte devices have been tested, but they require high temperatures for operation and often are moisture sensitive. Devices that measure pH changes when CO₂ adsorbs in a liquid solution have been proposed, but they are insensitive to low concentrations of CO₂. These sensors are presently either expensive compared to the sensor in a commercial carbon monoxide alarm or insufficiently sensitive.

FIG. 1 shows a typical prior art carbon monoxide detector such as that commonly used in a commercial carbon monoxide alarm. The design is similar to that disclosed in U.S. Pat. No. 6,948,352 (“the '352 patent”). The device consists of a membrane electrode assembly (MEA) that includes a Working Electrode 110, an ion conducting polymer membrane 111 and a Counter Electrode 112. The MEA is sandwiched between two hydrophobic current collectors 113 and 114. The device sits in a housing 115 with a water reservoir 116. There also is an insulating structure 117 to keep the anode from shorting to the housing.

During operation a voltage is applied between the two current collectors, 113 and 114. When the detector is exposed to carbon monoxide the carbon monoxide reacts on the Working Electrode 110 via the reaction:

CO+H₂O→CO₂+2H⁺+2e⁻

The protons (H⁺) travel through the membrane 211, and they react on the counter electrode via the reaction:

4H⁺+4e⁻+O₂→2H₂O

The net effect is that current is produced between the working electrode and counter electrode, wherein the amount of current is proportional to the carbon monoxide concentration.

Carbon monoxide sensors have the advantage that they are inexpensive to fabricate and there are well established techniques to mass produce them.

### SUMMARY OF THE INVENTION

The present carbon dioxide sensor design overcomes one or more of the limitations of high cost, moisture sensitivity and high temperature. The general approach is to create an electrochemical sensor with a working electrode, a counter electrode, and an electrolyte in between, wherein the electrochemical cell is active for CO₂ reduction to other chemicals. A voltage is then applied between the working electrode and the counter electrode. In one embodiment, the current produced is measured during the electrochemical reduction of CO₂ and uses that as a measure of the CO₂ concentration. In a second embodiment, the products of CO₂ reduction are allowed to build up in the cell and the concentration of the reaction products then measured either electrochemically or by other means.

Examples of reactions that can occur on the working electrode include:

CO₂+2e⁻→CO+½O₂²⁻

CO₂+2H⁺+2e⁻→CO+H₂O

CO₂+2H⁺+2e⁻→HCOOH

2CO₂+2e⁻→CO+CO₃²⁻

CO₂+H₂O+2e⁻→CO+2OH⁻

CO₂+2H₂O+4e⁻→HCO⁻+3OH

CO₂+2H₂O+2e⁻→H₂CO+2OH⁻

CO₂+H₂O+2e⁻→(HCO₂)⁻+OH⁻

CO₂+2H₂O+2e⁻→H₂CO₂+2OH⁻

CO₂+5H₂O+6e⁻→CH₃OH+6OH⁻

CO₂+6H₂O+8e⁻→CH₄+8OH⁻

CO₂+8H₂O+12e⁻→C₂H₄+12OH⁻

2CO₂+9H₂O+12e⁻→CH₃CH₂OH+12OH⁻

2CO₂+6H₂O+8e⁻→CH₃COOH+8OH⁻

2CO₂+5H₂O+8e⁻→CH₃COO⁻+7OH⁻

2CO₂+10H₂O+14e⁻→C₂H₆+14OH⁻

2CO₂+10H₂O+14e⁻→C₂H₆+14OH⁻

CO₂+2H⁺+2e⁻→CO+H₂O, acetic acid, oxalic acid, oxalate

CO₂+4H⁺+4e⁻→CH₄+O₂

where e⁻ is an electron. The examples given above are merely illustrative and are not meant to be an exhaustive list of all possible cathode reactions.

The present design can include a single cell as in FIG. 1. where CO₂ is converted, or multiple cells. In particular, the present design specifically includes devices with two electrochemical cells such as that illustrated in FIG. 2. In this case the electrochemical cell on the left is one electrochemical cell similar to that in FIG. 1, with a second electrochemical cell on the right. In the two-cell design illustrated in FIG. 2, the cell on the left-hand side contains a membrane electrode assembly (MEA) that includes a Working Electrode 110a, a proton conducting membrane 111a and a Counter Electrode 112a. The MEA is sandwiched between two hydrophobic current collectors 113a and 114a. The device sits in a housing 115a with a water reservoir 116a. There also is an insulating structure 117a to keep the anode from shorting to the housing. In the two-cell design illustrated in FIG. 2, the cell on the right-hand side contains a membrane electrode assembly (MEA) that includes a Working Electrode 110b, a proton conducting membrane 111b and a Counter Electrode 112b. The MEA is sandwiched between two hydrophobic current collectors 113b and 114b. The device sits in a housing 115b with a water reservoir 116b. There also is an insulating structure 117b to keep the anode from shorting to the housing. The two-cell embodiment can optionally include inlet 120, which permits CO₂ to enter the system and blocks water.

FIG. 2 shows two separate housings, but a single housing would be sufficient. For example, the Working Electrode could be held at negative potential while it is exposed to air or a gas mixture containing CO₂ so that one of the reaction products from the above-listed reactions builds up in the device. The Working Electrode could then be occasionally swept to positive potentials to determine how much of the product was created in the device.

Two Working Electrodes and a Counter Electrode could be contained in a single housing. The two electrodes could be interdigitated as illustrated in FIG. 3.

The present design is also employable in HVAC systems and patient monitors that include the sensors.

### BRIEF DESCRIPTION OF THE DRAWINGS

FIG. 1 is a schematic diagram of an electrochemical sensor in a conventional carbon monoxide alarm.

FIG. 2 is a schematic diagram of an exemplary dual electrode sensor for the detection of CO₂.

FIG. 3 is a schematic diagram of interdigitated electrodes.

FIGS. 4a, 4b and 4c illustrate cations that can be used to form a complex with (CO₂)⁻.

FIGS. 5a and 5b illustrate anions that can help to stabilize the (CO₂)⁻ anion.

FIG. 6 illustrates some of the neutral molecules that can be used to form a complex with (CO₂)⁻.

FIG. 7 is a schematic diagram of a cell used for the Examples set forth hereinafter.

FIG. 8 shows a comparison of the cyclic voltammetry for a blank scan where the catalyst was synthesized as in Example 1, where (i) a 99.9999% EMIM-BF4 solution was sparged with argon, and (ii) a scan where the same EMIM-BF4 solution was sparged with CO₂, in which platinum was employed as the catalyst. The large negative peak is associated with CO₂, and can be used to sense CO₂.

FIG. 9 shows a CO stripping experiment done by saturating a 99.9999% EMIM-BF4 solution with CO₂, holding the potential on a platinum catalyst at −0.845 V with respect to the standard hydrogen electrode (SHE) for 1, 5 or 10 minutes and then ramping the potential from −0.845 V to +2 V and measuring the current.

FIG. 10 shows a CO stripping experiment done by saturating a 98.55% EMIM-BF4 and 0.45% water solution with CO₂, holding the potential on a platinum catalyst at −0.6 V with respect to SHE for 1, 5 or 10 minutes and then ramping the potential from −0.6 to +2 V and measuring the current.

FIG. 11 shows a comparison of the cyclic voltammetry for a (i) blank scan where the catalyst was synthesized as in Example 4 where a 15% EMIM-BF4 in water solution was sparged with argon, and (ii) a scan where the same solution was sparged with CO₂. This experiment employed a silver catalyst.

FIG. 12 shows a CO stripping experiment done by saturating a 15% EMIM-BF4 in water solution with CO₂, holding the potential on a silver catalyst at −0.8 V with respect to SHE for 20 minutes and then ramping the potential from −0.6 to +2V and measuring the current.

FIG. 13 shows the results of a CO stripping experiment done with two Working Electrodes: a silver electrode that is held at −3.0 V with respect to the Counter Electrode, and a platinum Working Electrode that is swept between −0.1 and +0.3 V with respect to a silver reference electrode. The plot is the current at 0.17 V as a function of CO₂ concentration in the gas phase.

FIG. 14 is a comparison of the cyclic voltammetry for (i) a blank scan where the catalyst was synthesized as in Example 7 where the water-choline iodide mixture was sparged with argon and (ii) a scan where the water-choline iodide mixture was sparged with CO₂.

FIG. 15 shows a comparison of the cyclic voltammetry for (i) a blank scan where the catalyst was synthesized as in Example 8 where the water-choline chloride mixture was sparged with argon and (ii) a scan where the water-choline chloride mixture was sparged with CO₂.

FIG. 16 shows a CO stripping experiment done by starting with a catalyst mixture where the catalyst was synthesized as in Example 8 saturating the choline chloride solution with CO₂, holding the potential of the palladium Working Electrode at −0.6 V with respect to SHE for 20 minutes and then ramping the potential from −0.6 to +2 V and measuring the current.

FIG. 17 shows a comparison of the cyclic voltammetry for (i) a blank scan where the catalyst was synthesized as in Example 10 where the water-choline chloride mixture was sparged with argon and (ii) a scan where the water-choline chloride mixture was sparged with CO₂.

FIG. 18 shows a CO stripping experiment done by starting with a catalyst mixture where the catalyst was synthesized as in Example 10, saturating the choline chloride solution with CO₂, holding the potential of the nickel Working Electrode at −0.6 V with respect to SHE for 20 minutes, and then ramping the potential from −0.6 to +2 V and measuring the current.

### DETAILED DESCRIPTION OF PREFERRED EMBODIMENT(S)

It is understood that the invention is not limited to the particular methodology, protocols, and reagents described herein, as these may vary as the skilled artisan will recognize. It is also to be understood that the terminology used herein is used for the purpose of describing particular embodiments only, and is not intended to limit the scope of the invention. It also is to be noted that as used herein and in the appended claims, the singular forms “a,” “an,” and “the” include the plural reference unless the context clearly dictates otherwise. Thus, for example, a reference to “a linker” is a reference to one or more linkers and equivalents thereof known to those familiar with the technology involved here.

Unless defined otherwise, technical and scientific terms used herein have the same meanings as commonly understood by persons of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment can be employed with other embodiments as the skilled artisan would recognize, even if not explicitly stated herein.

Numerical value ranges recited herein include values from the lower value to the upper value in increments of one unit provided that there is a separation of at least two units between the lower value and the higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, and so on, are expressly enumerated in this specification. For values which are less than one, one unit is considered to be 0.0001, 0.001, 0.01 or 0.1 as appropriate. These are only examples of what is specifically intended and combinations of numerical values between the lowest value and the highest value are to be treated in a similar manner.

Moreover, provided immediately below is a “Definition” section, where certain terms related to the invention are defined specifically. Particular methods, devices, and materials are described, although methods and materials similar or equivalent to those described herein can be used in the practice or testing of the invention. References referred to herein are each incorporated by reference herein in its entirety, to the extent that they are not inconsistent with the present disclosure.

### DEFINITION

The term “commercial carbon monoxide alarm” refers to a commercial device that is able to monitor the concentration of carbon monoxide and sound an alarm if a threshold concentration is reached.

The term “electrochemical conversion of CO₂ as used here refers to electrochemical process where carbon dioxide, carbonate, or bicarbonate is converted into another chemical substance in a step of the process.

The term “CV” as used here refers to a cyclic voltammogram or cyclic voltammetry.

The term “Overpotential” as used here refers to the potential (voltage) difference between a reaction's thermodynamically determined reduction or oxidation potential and the potential at which the event is experimentally observed.

The term “Cathode Overpotential” as used here refers to the overpotential on the cathode of an electrochemical cell.

The term “Anode Overpotential” as used here refers to the overpotential on the anode of an electrochemical cell.

The term “Electron Conversion Efficiency” refers to selectivity of an electrochemical reaction. More precisely, it is defined as the fraction of the current that is supplied to the cell that goes to the production of a desired product.

The term “Catalytically Active Element” as used here refers to any chemical element that can serve as a catalyst for the electrochemical conversion of CO₂.

The term “Helper Catalyst”, as described in co-owned U.S. Pat. No. 8,956,990, hereby incorporated by reference in its entirety, refers to an organic molecule, organic ion, salt of an organic ion, or mixture of such organic molecules, ions, and/or salts that does at least one of the following:
Speeds up a chemical reaction, or
Lowers the overpotential of the reaction without being substantially consumed in the process.

The term “Active Element, Helper Catalyst Mixture” refers to a mixture that includes one or more Catalytically Active Element(s) and, separately, at least one Helper Catalyst.

The term “Ionic Liquid” refers to salts or ionic compounds that form stable liquids at temperatures below 200° C.

The term “Deep Eutectic Solvent” refers to an ionic solvent that includes a mixture which forms a eutectic with a melting point lower than that of the individual components.

The term “Director Substance” (or “Director Ion” or “Director Molecule”) refers to a molecule, ion or substance that increases the selectivity of a reaction. If a Director Substance is added to a reaction mixture, the selectivity for a desired reaction goes up. This effect may be the result of suppressing undesired side reactions, or blocking the adsorption of some species even if the desired reaction is also slowed, as long as the selectivity toward the desired reaction is increased.

The term “Hydrogen Suppressor” refers to a molecule that either: (a) decreases the rate of hydrogen formation, or (b) increases the overpotential for hydrogen formation, when the molecule is added to a reaction mixture.

The term “MEA” refers to a membrane electrode assembly that includes a working electrode, a counter electrode and an ion conducting membrane.

The term “Working Electrode” refers to the electrode in an electrochemical system on which the reaction of interest, such as conversion of CO₂, is occurring.

The term “Counter Electrode” refers to a secondary electrode in an electrochemical cell that is used to complete the electrochemical circuit so that current can flow through the device. Generally, a potential is applied between the working electrode and the counter electrode to allow the electrochemical reaction to occur.

The term “electrochemical cell” refers to a device with a working electrode, a counter electrode, and an electrolyte that can carry ions from the working electrode to the counter electrode.

The term “SHE” refers to the potential of a standard hydrogen electrode

The term “RHE” refers to the potential of a reversible hydrogen electrode

The term “Electrochemical Reaction” is a chemical reaction either caused or accompanied by the passage of an electric current and involving in most cases the transfer of electrons between an electrode and another substance.

The term “Electrochemical Reduction” is an electrochemical reaction where a species is chemically reduced.

The term “EMIM” refers to 1-ethyl-3-methylimidazolium cations.

The term “EMIM-BF4 refers to 1-ethyl-3-methylimidazolium tetrafluoroborate.

The term “HVAC” refers to a system for heating, ventilation and air conditioning.

The term “interdigitated” refers to an electrode arrangement where there are two electrodes that are not in direct electrical contact wherein the smallest rectangle enclosing the catalyst on one of the electrodes overlaps the smallest rectangle overlapping the catalyst on the second electrode when viewed perpendicularly to any point on the surface supporting either electrode. For this definition electrodes that are directly connected to one another are considered a single electrode.

The term “imidazolium” as used here refers to a positively charged ligand containing an imidazole group. This includes a bare imidazole or a substituted imidazole. Ligands of the form:

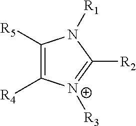

where R₁-R₅ are each independently selected from hydrogen, halogens, linear alkyls, branched alkyls, cyclic alkyls, heteroalkyls, aryls, cyclic aryls, heteroaryls, alkylaryls, heteroalkylaryls, and polymers thereof, such as the vinyl benzyl copolymers described herein, are specifically included.

The term “pyridinium” as used here refers to a positively charged ligand containing a pyridinium group. This includes a protonated bare pyridine or a substituted pyridine or pyridinium. Ligands of the form

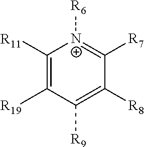

where R₆-R₁₁ are each independently selected from hydrogen, halogens, linear alkyls, branched alkyls, cyclic alkyls, heteroalkyls, aryls, cyclic aryls, heteroaryls, alkylaryls, heteroalkylaryls, and polymers thereof, such as the vinyl benzyl copolymers described herein, are specifically included.

The term “pyrazoliums” as used here refers to a positively charged ligand containing a pyrazolium group. This includes a bare pyrazolium or a substituted pyrazolium. Ligands of the form

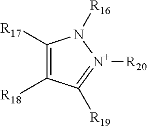

where R₁₆-R₂₀ are each independently selected from hydrogen, halogens, linear alkyls, branched alkyls, cyclic alkyls, heteroalkyls, aryls, cyclic aryls, heteroaryls, alkylaryls, heteroalkylaryls, and polymers thereof, such as the vinyl benzyl copolymers described herein, are specifically included.

The term “positively charged cyclic amine” as used here refers to a positively charged ligand containing a cyclic amine. This specifically includes imidazoliums, pyridiniums, pyrazoliums, pyrrolidiniums, pyrroliums, pyrimidiums, piperidiniums, indoliums, triaziniums, and polymers thereof, such as the vinyl benzyl copolymers described herein.

### Specific Description

The present invention relates generally to a CO₂ sensor design that includes an electrochemical cell that converts carbon dioxide into another substance when a sufficient voltage is applied. For example, the working electrode of the electrochemical cell may be active for CO₂ reduction reactions such as:

CO₂+2e⁻→CO+½O₂²⁻

CO₂+2H⁺+2e⁻→CO+H₂O

CO₂+2H⁺+2e⁻→HCOOH

2CO₂+2e⁻→CO+CO₃²⁻

CO₂+H₂O+2e⁻→CO+2OH⁻

CO₂+2H₂O+4e⁻→HCO⁻+3OH

CO₂+2H₂O+2e⁻→H₂CO+2OH⁻

CO₂+H₂O+2e⁻→(HCO₂)−+OH⁻

CO₂+2H₂O+2e⁻→H₂CO₂+2OH⁻

CO₂+5H₂O+6e⁻→CH₃OH+6OH⁻

CO₂+6H₂O+8e⁻→CH₄+8OH⁻

CO₂+8H₂O+12e⁻→C₂H₄+12OH⁻

2CO₂+9H₂O+12e⁻→CH₃CH₂OH+12OH⁻

2CO₂+6H₂O+8e⁻→CH₃COOH+8OH⁻

2CO₂+5H₂O+8e⁻→CH₃COO−+7OH⁻

2CO₂+10H₂O+14e⁻→C₂H₆+14OH⁻

CO₂+2H⁺+2e⁻→CO+H₂O, acetic acid, oxalic acid, oxalate

CO₂+4H⁺+4e⁻→CH₄+O₂

where e⁻ is an electron. The examples given above are merely illustrative and are not meant to be an exhaustive list of all possible reactions in the electrochemical cell.

When the above reactions occur, current flows between the working electrode and the counter electrode. In all cases the current changes as the CO₂ concentration changes. As a result, a sensor with a working electrode that is active for one of the above-listed reactions could be used as a CO₂ sensor.

Working electrodes for CO₂ conversion are disclosed in U.S. Pat. Nos. 3,959,094; 4,240,882; 4,523,981; 4,545,872; 4,595,465; 4,608,132; 4,608,133; 4,609,440; 4,609,441; 4,609,451; 4,620,906; 4,668,349; 4,673,473; 4,711,708; 4,756,807; 4,818,353; 5,064,733; 5,284,563; 5,382,332; 5,457,079; 5,709,789; 5,928,806; 5,952,540; 6,024,855; 6,660,680; 6,987,134; 7,157,404; 7,378,561; 7,479,570; U.S. Patent Application Publication No. US200810223727, and the papers reviewed by Hori (Modern Aspects of Electrochemistry, 42, 89-189, 2008) (“the Hori Review”), Gattrell, et al. (Journal of Electroanalytical Chemistry, 594, 1-19, 2006) (“the Gattrell review”), and DuBois (Encyclopedia of Electrochemistry, 7a, 202-225, 2006) (“the DuBois review”). The Working Electrodes include one or more of the following Catalytically Active Elements: V, Cr, Mn, Fe, Co, Ni, Cu, Sn, Zr, Nb, Mo, Ru, Rh, Pd, Ag, Cd, Hf, Ta, W, Re, Ir, Pt, Au, Hg, Al, Si, In, Tl, Pb, Bi, Sb, Te, U, Sm, Tb, La, Ce, and Nd Sensors using the working electrodes described in these papers, patents and patent applications are included in the invention.

The preferred design is a CO₂ sensor that is able to determine the CO₂ concentration in the presence of water vapor. All of the working electrodes disclosed in U.S. Pat. Nos. 3,959,094; 4,240,882; 4,523,981; 4,545,872; 4,595,465; 4,608,132; 4,608,133; 4,609,440; 4,609,441; 4,609,451; 4,620,906; 4,668,349; 4,673,473; 4,711,708; 4,756,807; 4,818,353; 5,064,733; 5,284,563; 5,382,332; 5,457,079; 5,709,789; 5,928,806; 5,952,540; 6,024,855; 6,660,680; 6,987,134; 7,157,404; 7,378,561; 7,479,570; U.S. Patent Application Publication No. US2008/0223727, and the papers reviewed in the Hori Review, the Gattrell review and the DuBois review are also active for the electrolysis of water and in most cases the rate of water electrolysis is much larger than the rate of CO₂ conversion. Consequently, an MEA made using the catalysts and methods disclosed in U.S. Pat. Nos. 3,959,094; 4,240,882; 4,523,981; 4,545,872; 4,595,465; 4,608,132; 4,608,133; 4,609,440; 4,609,441; 4,609,451; 4,620,906; 4,668,349; 4,673,473; 4,711,708; 4,756,807; 4,818,353; 5,064,733; 5,284,563; 5,382,332; 5,457,079; 5,709,789; 5,928,806; 5,952,540; 6,024,855; 6,660,680; 6,987,134; 7,157,404; 7,378,561; 7,479,570; U.S. Patent Application Publication No. US2008/0223727 and papers reviewed by Hori (Modern Aspects of Electrochemistry, 42, pages 89-189, 2008) (“the Hori Review”), Gattrell, et al. (Journal of Electroanalytical Chemistry, 594, pages 1-19, 2006) (“the Gattrell review”), or DuBois (Encyclopedia of Electrochemistry, 7a, pages 202-225, 2006) (“the DuBois review”), will show an extra current in the presence of water vapor and therefore will not provide a quantitative measurement of the CO₂ concentration in the presence of water vapor. This is not preferred.

Generally, the preferred devices can detect CO₂ in the presence of water vapor. They will include an electrochemical cell with a Working Electrode and a counter electrode with an electrolyte in between wherein the Working Electrode will include a Catalytically Active Element that is active for the electro-reduction of CO₂. The preferred devices can also include at least one of (i) Helper Catalysts, (ii) Director Substances, and (iii) Hydrogen Suppressors to enhance the current due to CO₂ conversion and/or to reduce the current due to the electrolysis of water. Generally, the Helper Catalyst will serve to enhance the rate of CO₂ conversion so that the current due to CO₂ conversion is increased. The Director Substances will improve the selectivity of the electrochemical cell, so that CO₂ is preferentially reduced. The Hydrogen Suppressor will specifically act to inhibit the electrolysis of water.

Exemplary Catalytically Active Elements include: V, Cr, Mn, Fe, Co, Ni, Cu, Sn, Zr, Nb, Mo, Ru, Rh, Pd, Ag, Cd, Hf, Ta, W, Re, Ir, Pt, Au, Hg, Al, Si, In, Sn, Tl, Pb, Bi, Sb, Te, U, Sm, Tb, La, Ce, and Nd, but the invention is not limited to this list of chemical elements.

The Helper Catalyst is a substance that lowers the overpotential for CO₂ electrolysis. For example, the Helper Catalyst may adsorb on the Working Electrode in the sensor, and modify the electrode's electrochemical behavior so that the overpotential for CO₂ reduction is decreased.

According to Chandrasekaran, et al. (Surface Science, 185, 495-514, 1987), the high overpotentials for CO₂ conversion occur because the first step in the electroreduction of CO₂ is the formation of a CO₂⁻ intermediate. It takes energy to form the intermediate. This results in a high overpotential for the reaction.

In principle, the overpotential can be reduced by identifying a substance, designated HPER, that can bind to the CO₂⁻ intermediate on or near the Catalytically Active Element. When a bond is formed between the CO₂⁻ and the substance HPER, the free energy of formation of the CO₂⁻ will also be lowered. Consequently, the energy needed to form the CO₂⁻ will be reduced. Therefore, the substance HPER can act as a Helper Catalyst.

Previous literature indicates that solutions including one or more of: ionic liquids, deep eutectic solvents, amines, and phosphines, including specifically imidazoliums (also called imidazoniums), pyridiniums, pyrrolidiniums, phosphoniums, ammoniums, sulfoniums, prolinates, and methioninates can form complexes with CO₂. Consequently, they can serve as Helper Catalysts. Also Davis Jr., et al. (In ACS Symposium Series 856: Ionic Liquids as Green Solvents: Progress and Prospects, pages 100-107, 2003) list a number of other salts that show ionic properties. Specific examples include compounds including one or more of acetocholines (also called acetylcholines), alanines, aminoacetonitriles, methylammoniums, arginines, aspartic acids, cholines, threonines, chloroformamidiniums, thiouroniums, quinoliniums, pyrrolidinols, serinols, benzamidines, sulfamates, acetates, carbamates, triflates, and cyanides. These salts can act as Helper Catalysts. These examples are meant for illustrative purposes only, and are not meant to limit the scope of the invention.

Of course, not every substance that forms a complex with (CO₂)⁻ acts as a Helper Catalyst. Masel (Chemical Kinetics and Catalysis, Wiley 2001, pages 717-720), notes that when an intermediate binds to a catalyst, the reactivity of the intermediate decreases. If the intermediate bonds too strongly to the catalyst, the intermediate will become unreactive, so the substance will not be effective. This provides an important limitation on substances that act as Helper Catalysts. The Helper Catalyst cannot form so strong of a bond with the (CO₂)⁻ that the (CO₂)⁻ is unreactive toward the Catalytically Active Element.

More specifically, the substance should form a complex with the (CO₂)⁻ so that the complex is stable (that is, has a negative free energy of formation) at potentials less negative than −1 V with respect to the standard hydrogen electrode (SHE). However, the complex should not be so stable that the free energy of the reaction between the complex and the Catalytically Active Element is more positive than about 5 kcal/mol.

Those trained in the state of the art should realize that the ability of the Helper Catalyst to stabilize the (CO₂)⁻ also varies with the anion. For example, Zhao, et al. (The Journal of Supercritical Fluids, 32, 287-291, 2004) examined CO₂ conversion in 1-n-butyl-3-methylimidazolium hexafluorophosphate (BMIM-PF6), but FIG. 3 in Zhao, et al., shows that the BMIM-PF6 did NOT lower the overpotential for the reaction (that is, the BMIM-PF6 did not act as a Helper Catalyst). This may be because the BMIM-PF6 formed such a strong bond to the (CO₂)⁻ that the CO₂ was unreactive with the copper. Similarly, Yuan, et al., Electrochimica Acta 54 (2009) pages 2912-2915, examined the reaction between methanol and CO₂ in 1-butyl-3-methylimidazolium bromide (BMIM-Br). The BMIM-Br did not act as a Helper Catalyst. This may be because the complex was too weak or that the bromine poisoned the reaction.

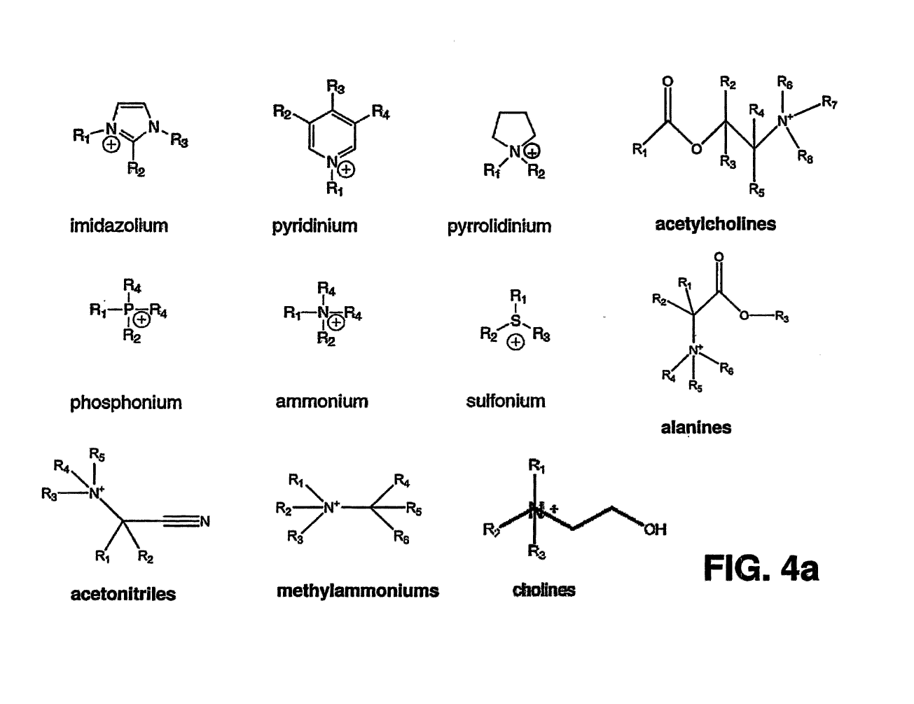

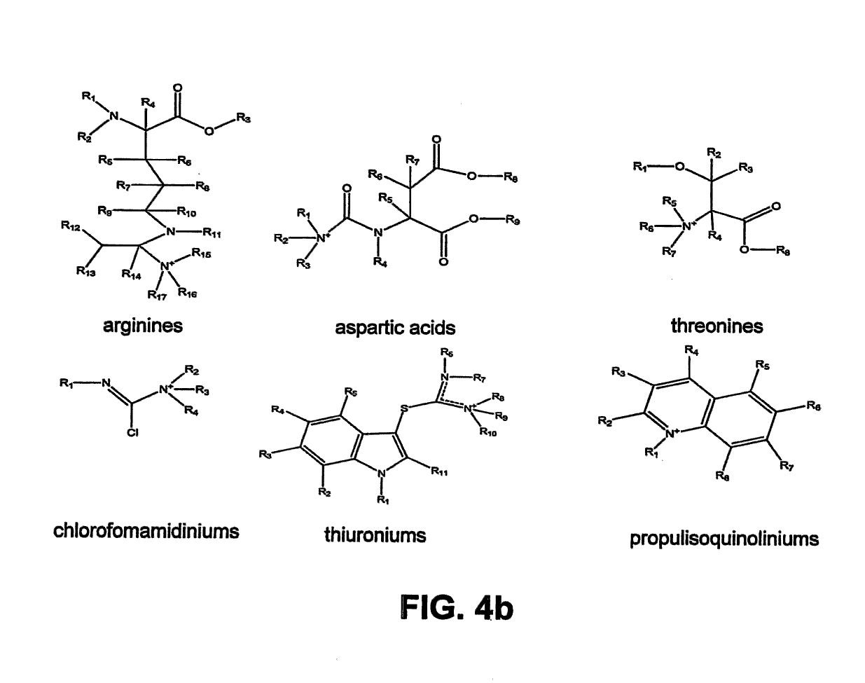

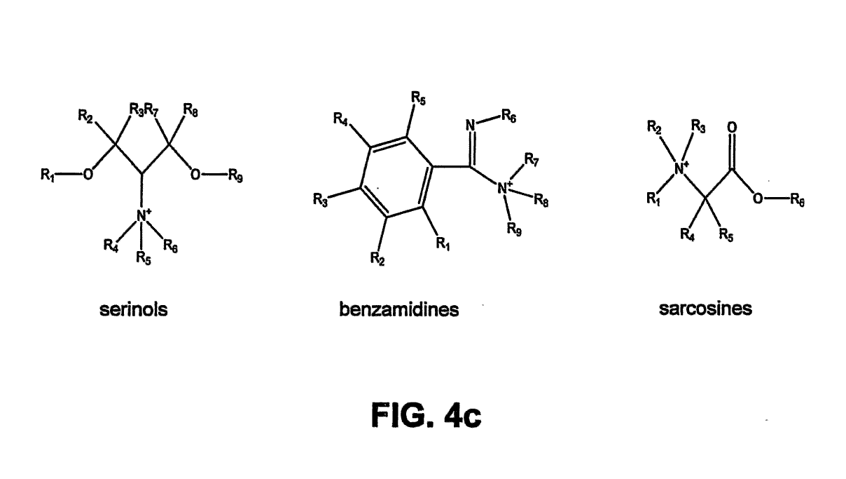

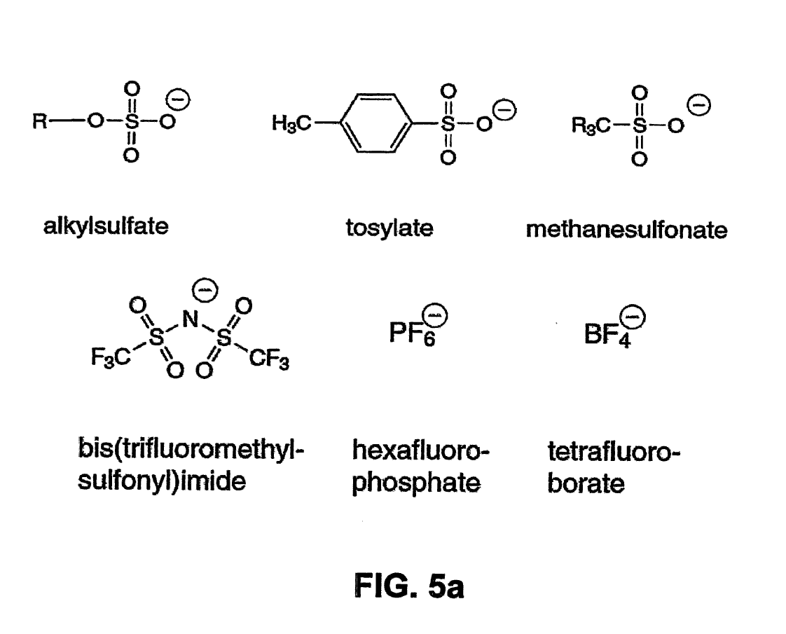

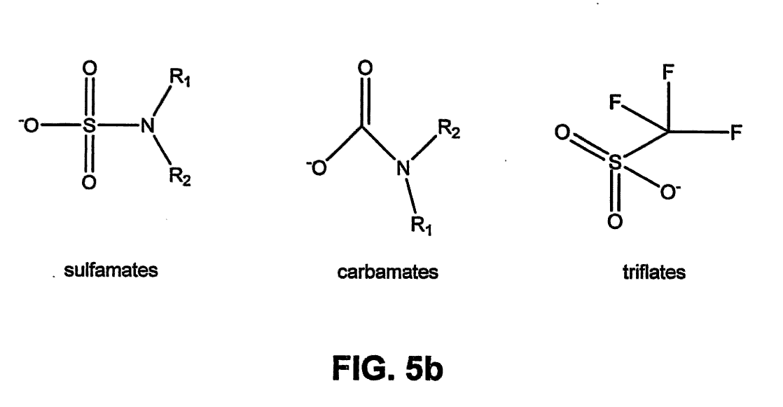

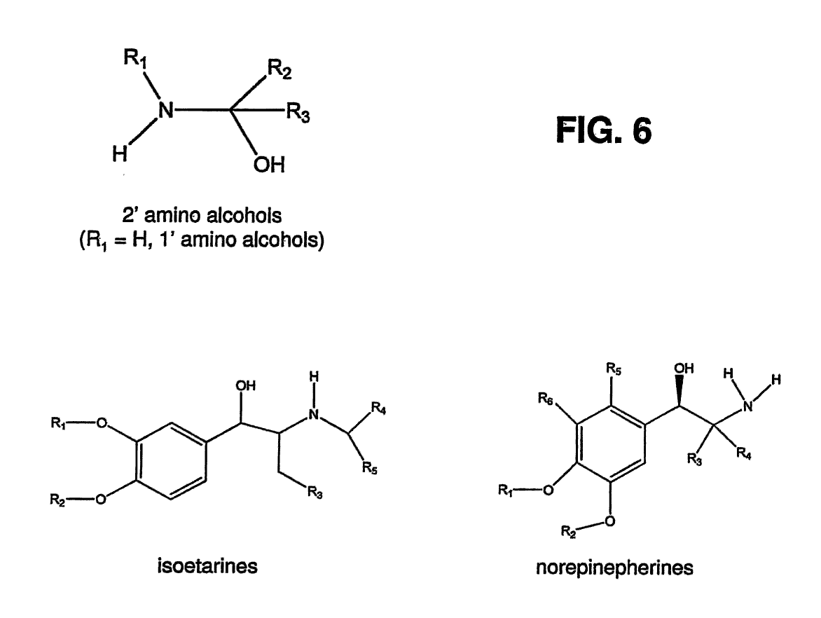

Solutions that include one or more of the cations in FIGS. 4a, 4b and 4c, as well as the anions in FIGS. 5a and 5b, and/or the neutral species in FIG. 6, where R₁, R₂ and R₃ (and R₄-R₁₇) include H, OH or a ligand containing at least one carbon atom, are believed to form complexes with CO₂ or (CO₂)⁻. They all can be Helper Catalysts but are not necessarily Helper Catalysts.

Specific examples include: imidazoliums (also called imidazoniums), pyridiniums, pyrrolidiniums, phosphoniums, ammoniums, sulfoniums, prolinates, and methioninates. All of these examples might be able to be used as Helper Catalysts for CO₂ conversion, and are specifically included in the invention. These examples are meant for illustrative purposes only, and are not meant to limit the scope of the invention.

Whether a given substance S is a Helper Catalyst for a reaction R or a Working Electrode M can be determined as follows:

Fill a standard 3 electrode electrochemical cell with the electrolyte commonly used for reaction R. Common electrolytes such as 0.1 M sulfuric acid or 0.1 M KOH in water can also be used.

Mount the Working Electrode into the 3 electrode electrochemical cell and provide an appropriate counter electrode.

Run several CV cycles to clean the active metal.

Measure RHE potential in the electrolyte.

Load the reactants for the reaction R into the cell, and measure a CV of the reaction R, noting the potential of the peak associated with the reaction R.

Calculate V1=the difference between the onset potential of the peak associated with reaction and RHE.

Calculate V1A=the difference between the maximum potential of the peak associated with reaction and RHE.

Add 0.0001 to 99.9999% of the substance S to the electrolyte.

Measure RHE in the reaction with Helper Catalyst.

Measure the CV of reaction R again, noting the potential of the peak associated with the reaction R.

Calculate V2=the difference between the onset potential of the peak associated with reaction R and RHE.

Calculate V2A=the difference between the maximum potential of the peak associated with reaction R and RHE.

If V2<V1 or V2A<V1A at any concentration of the substance S between 0.0001 and 99.9999%, the substance S is a Helper Catalyst for the reaction.

The substance S will also be a Helper Catalyst if the replacement of the original electrolyte by a solution of the substance S in water or other appropriate solvent results in V2<V1 or V2A<V1A.

Further, the Helper Catalyst could be in one of the following forms: (i) a solvent for the reaction; (ii) an electrolyte; (iii) an additive to a component of the system, or (iv) something that is bound to at least one of the catalysts in a system. These examples are meant for illustrative purposes only, and are not meant to limit the scope of the invention.

Hydrogen Suppressors act in the opposite way as the Helper Catalysts. In particular, a Hydrogen Suppressors can raise the overpotential for water electrolysis to hydrogen. The Hydrogen Suppressor can adsorb onto the Working Electrode, and either repel protons or block hydrogen adsorption onto the Working Electrode.

An example of such a Hydrogen Suppressor would be a salt including the choline cation, or a choline derivative of the form R₁R₂R₃N⁺(CH₂)_{N}OH, R₁R₂R₃N⁺(CH₂)ₙCOH or R₁R₂R₃N⁺(CH₂)ₙCOOH wherein n=1-4, and R₁, R₂ and R₃ are each a ligand containing at least 1 carbon atom. Preferably, R₁, R₂ and R₃ are independently selected from the group consisting of aliphatic C₁-C₄ groups, —CH₂OH, —CH₂CH₂OH, —CH₂CH₂CH₂OH, —CH₂CHOHCH₃, —CH₂COH, —CH₂CH₂COH, and —CH₂COCH₃ and molecules where one of more chlorine or fluorine is substituted for the hydrogens in aliphatic C₁-C₄ groups, —CH₂OH, —CH₂CH₂OH, —CH₂CH₂CH₂OH, —CH₂CHOHCH₃, —CH₂COH, —CH₂CH₂COH, and —CH₂COCH₃. These examples are meant for illustrative purposes only, and are not meant to limit the scope of the invention.

Choline chlorine, tetrabutylammonium hydrogen sulfate (TBAHS) and ethylenediaminetetraacetic acid (EDTA) are specifically included as Hydrogen Suppressors in the present design. These examples are meant for illustrative purposes only, and are not meant to limit the scope of the invention.

The Hydrogen Suppressors can also include benzaldehyde and substituted benzaldehydes and di-acids such as succinic acid and substituted di-acids. These examples are meant for illustrative purposes only, and are not meant to limit the scope of the invention.

Again, not every R₁R₂R₃N⁺(CH₂)ₙOH, R₁R₂R₃N⁺(CH₂)ₙCOH or R₁R₂R₃N⁺(CH₂)ₙCOOH will suppress hydrogen formation, and the effectiveness of the Hydrogen Suppressor varies with the cathode metal. Whether a given substance is a Hydrogen Suppressor can be determined for a Working Electrode that includes a Catalytically Active Element M as follows:

Fill a standard 3 electrode electrochemical cell with the electrolyte with a pH similar to that of the substance S. A standard electrolyte such as 0.1 M sulfuric acid or 0.1 M sodium hydroxide can also be used.

Mount the active metal on the Working Electrode in the 3 electrode electrochemical cell and provide an appropriate counter electrode.

Run several CV cycles to clean the active metal.

Measure the reversible hydrogen electrode (RHE) potential in the electrolyte.

Add water to the cell if not already present, and measure a CV of the reaction, noting the potential of the peak associated with hydrogen formation.

Calculate V1=the difference between the onset potential of the peak associated with hydrogen formation and RHE.

Calculate V1A=the difference between the maximum potential of the peak associated with reaction and RHE.

Add 0.0001 to 99.9999% of the substance S to the electrolyte.

Measure RHE in the cell with substance S present.

Measure the CV of the cell again, noting the potential of the peak associated with hydrogen evolution. If no hydrogen evolution peak is seen, the substance S is a Hydrogen Suppressor

If a hydrogen evolution peak is seen, calculate V2=the difference between the onset potential of the peak associated with hydrogen evolution and RHE.

Calculate V2A=the difference between the maximum potential of the peak associated with the hydrogen evolution reaction and RHE.

If V2>V1 or V2A>V1A at any concentration of the substance S between 0.0001 and 99.9999%, the substance S is a Hydrogen Suppressor for that catalyst.

The substance S is also a Hydrogen Suppressor if the original electrolyte in the cell above can be substituted with a solution containing 0.0001 to 99.9999% of the Helper Catalyst and V2>V1 or V2A>V1A at any concentration of the substance S between 0.0001 and 99.9999%.

Further, the Hydrogen Suppressor could be in one of the following forms: (i) a solvent for the reaction; (ii) an electrolyte; (iii) an additive to a component of the system; or (iv) a constituent that is bound to at least one of the catalysts in a system. These examples are meant for illustrative purposes only, and are not meant to limit the scope of the present invention.

Director Substances are a molecule or an ion that improves the selectivity of the sensor. Director Substances can include Helper Catalysts, Hydrogen Suppressors, or substances that enhance the solubility of CO₂. A simple test for a Director Substance D is to:

Measure the current that the sensor produces when exposed to a) air that is saturated with water and b) air that has 1500 ppm of CO₂ in it.

Add the substance D to the sensor and measure the current under both of the above conditions again.

If the current due to CO₂ goes up or the current in the presence of water goes down, the substance D will be a Director Substance.

Examples of Director Substances include imidazoliums (also called imidazoniums), pyridiniums, pyrrolidiniums, phosphoniums, ammoniums, sulfoniums, prolinates, methioninates, acetocholines (also called acetylcholines), alanines, aminoacetonitriles, methylammoniums, arginines, aspartic acids, cholines, threonines, chloroformamidiniums, thiouroniums, quinoliniums, pyrrolidinols, serinols, benzamidines, sulfamates, acetates, carbamates, triflates, and cyanides, amines, phosphonates, polyimides, other nitrogen or phosphorous containing polymers, and anion exchange resins. These examples are meant for illustrative purposes only, and are not meant to limit the scope of the invention.

Those familiar with the technology involved here should recognize that only a tiny amount of the Helper Catalyst, Hydrogen Suppressors and/or Director Substances may be needed to have a significant effect. Catalytic reactions often occur on distinct active sites. The active site concentration can be very low, so in principle a small amount of Helper Catalyst, Hydrogen Suppressors and/or Director Substances can have a significant effect on the rate. An estimate of how little of the Helper Catalyst would be needed to change the reaction can be obtained from the Pease et al., JACS 47, page 1235 (1925) study of the effect of carbon monoxide (CO) on the rate of ethylene hydrogenation on copper. This paper is incorporated herein by reference in its entirety. Pease et al. found that 0.05 cc's (62 micrograms) of carbon monoxide (CO) was sufficient to almost completely poison a 100 gram catalyst towards ethylene hydrogenation. This corresponds to a poison concentration of 0.0000062% by weight of CO in the catalyst. Those trained in the state of the art know that if 0.0000062% by weight of the poison in a Catalytically Active Element-poison mixture could effectively suppress a reaction, then as little as 0.0000062% by weight of Helper Catalyst, Hydrogen Suppressors and/or Director Substances could also prevent a side reaction. This provides an estimate of a lower limit to the Helper Catalyst, Hydrogen Suppressors and/or Director Substance concentration in the invention.

The upper limit is illustrated in Example 1 below, where the Active Element, Helper Catalyst Mixture could have approximately 99.999% by weight of Helper Catalyst, and the Helper Catalyst could be at least an order of magnitude more concentrated. Thus the range of Helper Catalyst concentrations for the present design can be 0.0000062% to 99.9999% by weight.

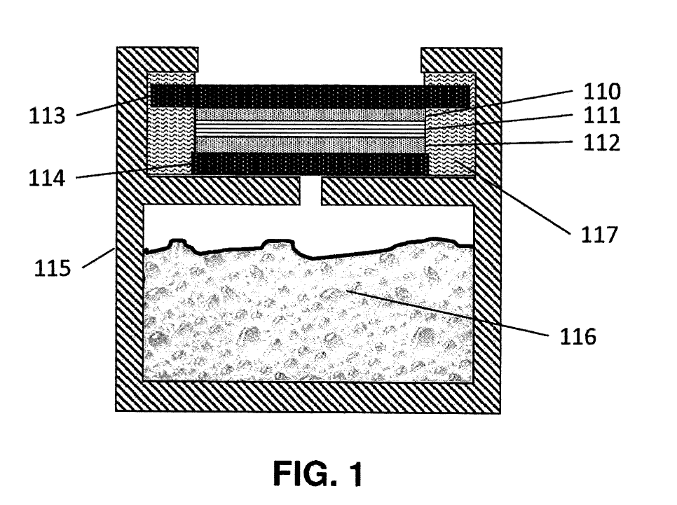

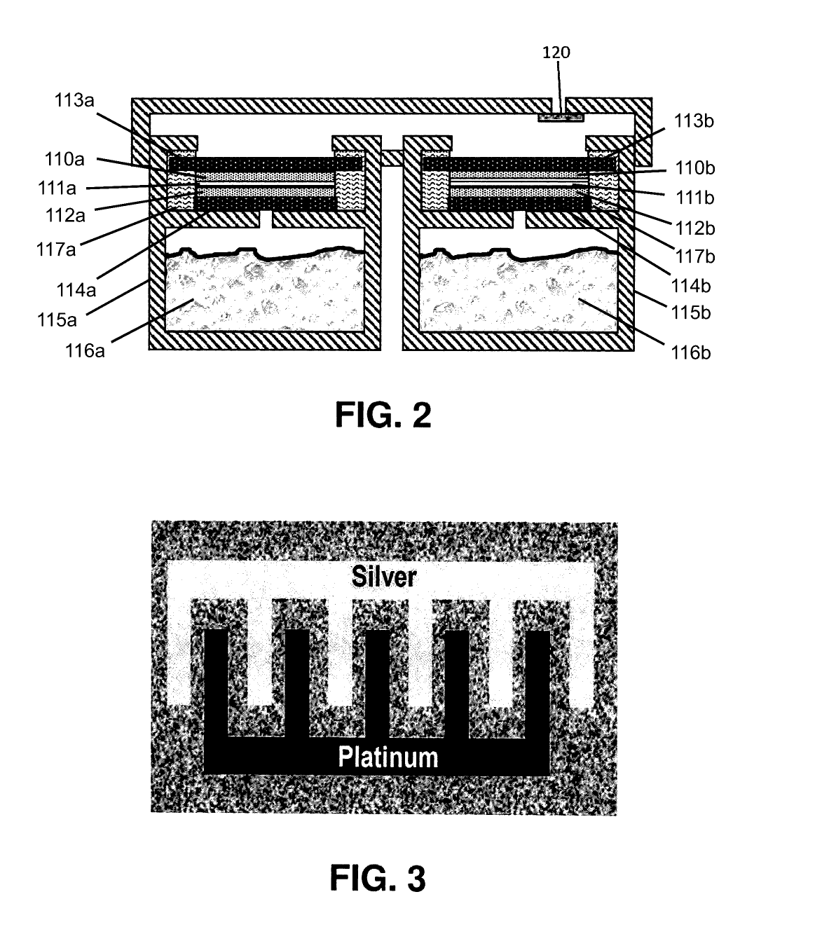

The complete sensor will include one or more Working Electrodes, at least one counter electrode, and an electrolyte in a housing. Examples include FIG. 1 and FIG. 2. The present technique specifically contemplates devices with two or more interdigitated electrodes. These examples are meant for illustrative purposes only, and are not meant to limit the scope of the invention.

The present design also includes systems that include the sensors described here. HVAC systems, systems to monitor patients, systems to treat patients and systems to measure CO₂ concentrations in liquids are also specifically included. The latter system will include a membrane to separate the liquid from the Working Electrode. These examples are meant for illustrative purposes only, and are not meant to limit the scope of the invention.

Without further elaboration, it is believed that persons familiar with the technology involved here using the preceding description can utilize the invention to the fullest extent. The following examples are illustrative only, and are not meant to be an exhaustive list of all possible embodiments, applications or modifications of the invention.

### EXAMPLE 1

A CO₂ sensor with a Working Electrode including an Active Element, Helper Catalyst Mixture including platinum and 1-ethyl-3-methylimidazolium tetrafluoroborate (EMIM-BF4)

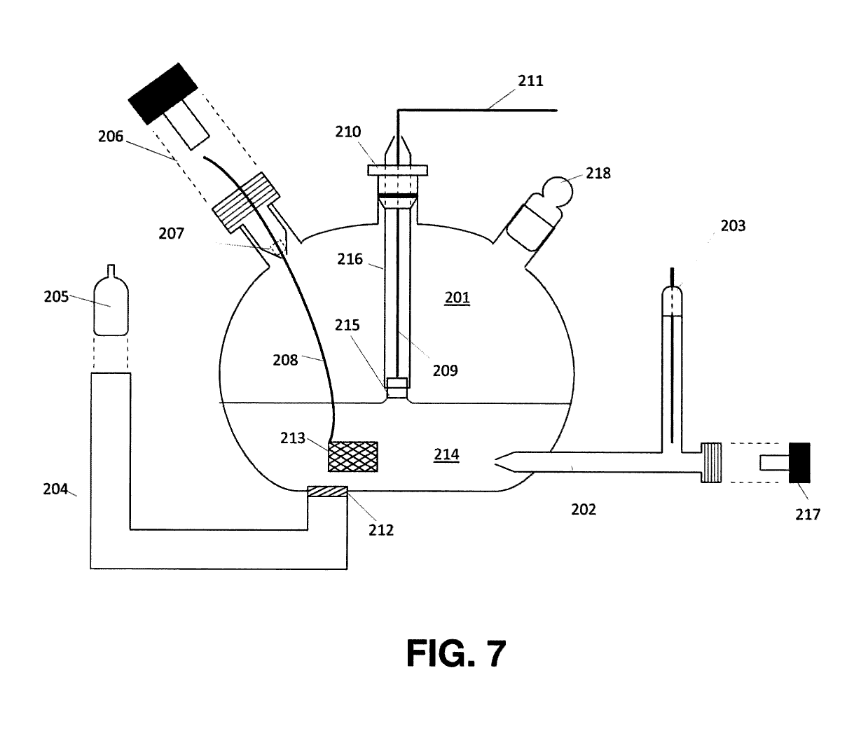

The experiments used the glass three electrode cell shown in FIG. 7. The cell consisted of a three neck flask 201, to hold the anode 213, the gold cathode 215, and the ionic liquid solution 214. Seal 207 forms a seal around anode wire 208. Fitting 206 compresses seal 207 around anode wire 208. Rotary seal 210 facilitates rotation of shaft 216, which in turn causes gold plug 215 to spin. Wire 209 and contact 211 allow a connection to be made to the cathode. Seal 218 closes the unused third neck of flask 201. CO₂ enters the system through a glass connector 205, through a tube 204 and a frit 212.

A silver/0.01 molar silver ion reference electrode 203 in acetonitrile was connected to the cell through a Luggin Capillary 202, which includes a seal 217. The reference electrode 203 was fitted with a Vycor® frit to prevent the reference electrode solution from contaminating the ionic liquid in the capillary. The reference electrode was calibrated against the ferrocene Fc/Fc+ redox couple. A conversion factor of +535 was used to convert our potential axis to reference the Standard Hydrogen Electrode (SHE). A 25×25 mm platinum gauze 213 (size 52) was used as an anode while a 0.33 cm² polycrystalline gold plug 215 was used as the cathode.

Prior to the experiments all glass parts were put through a 1% Nochromix® bath (2 hours), followed by a 50/50 v/v nitric acid/water bath (12 hours), followed by rinsing with Millipore-filtered water. In addition, the gold plug 215 and platinum gauze 213 were mechanically polished using procedures known to workers trained in the art. They were then cleaned in a sulfuric acid bath for 12 hours.

During the experiment a catalyst ink including a Catalytically Active Element, platinum, was first prepared as follows: First, 0.056 grams of Johnson-Matthey Hispec 1000 platinum black purchased from Alfa-Aesar was mixed with 1 gram of Millipore water and sonicated for 10 minutes to produce a solution containing a 5.6 mg/ml suspension of platinum black in Millipore water. A 25 μl drop of the ink was placed on the gold plug 215 and allowed to dry under a heat lamp for 20 min, and subsequently allowed to dry in air for an additional hour. This yielded a Working Electrode with 0.00014 grams of Catalytically Active Element, platinum, on a gold plug. The gold plug was mounted into the three neck flask 201. Next a Helper Catalyst, EMIM-BF4 (EMD Chemicals, Inc., San Diego, Calif., USA) was heated to 120° C. under a −23 in. Hg vacuum for 12 hours to remove residual water and oxygen. The concentration of water in the ionic liquid after this procedure was found to be approximately 90 mM by conducting a Karl-Fischer titration (that is, the ionic liquid contained 99.9999% of Helper Catalyst). 13 grams of the EMIM-BF4 was added to the vessel, creating an Active Element, Helper Catalyst Mixture that contained about 99.999% of the Helper Catalyst. The geometry was such that the gold plug formed a meniscus with the EMIM-BF4 Next ultra-high-purity (UHP) argon was fed through the sparging tube 204 and glass frit 212 for 2 hours at 200 sccm to further remove moisture picked up by contact with the air.

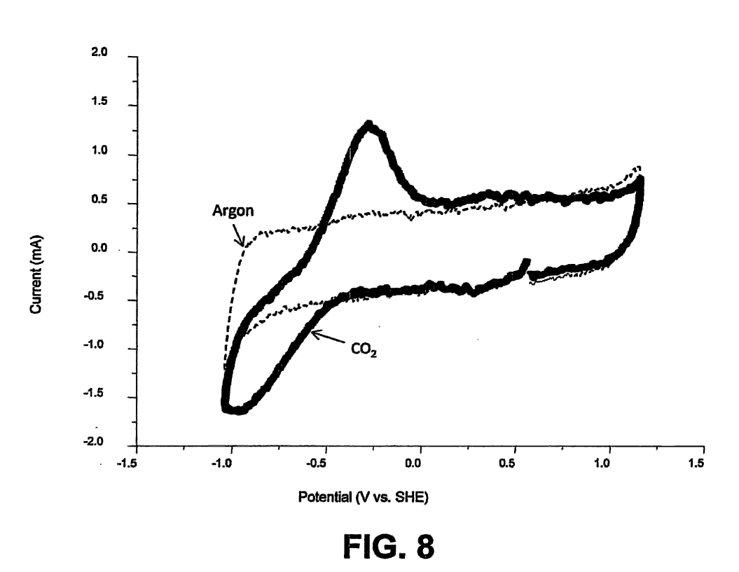

Next, the Working Electrode was connected to the Working Electrode connection in an SI 1287 Solartron electrical interface, the anode was connected to the Counter Electrode connection and the reference electrode was connected to the reference electrode connection on the Solartron. Then the potential on the cathode was held at −1.5 V versus a standard hydrogen electrode (SHE), raised to 1 V vs. SHE, and then scanned back to −1.5 volts versus SHE thirty times at a scan rate of 50 mV/s. The current produced during the last scan is labeled as the “argon” scan in FIG. 8.

Next, CO₂ was bubbled through the sparging tube at 200 sccm for 30 minutes, and the same scanning technique was used. That produced the CO₂ scan in FIG. 8. Notice the peak starting at −0.2 volts with respect to SHE, and reaching a maximum at −0.4 V with respect to SHE. That peak is associated with CO₂ conversion.

The peak can be used to detect the presence of CO₂. Consequently, a device with a Working Electrode including platinum and 1-ethyl-3-methylimidazolium tetrafluoroborate (EMIM-BF4) could be used as a CO₂ sensor.

### EXAMPLE 2

This example demonstrates an alternate operation mode for a CO₂ sensor where CO₂ is first converted to another substance and then detected. Specifically, in this example CO will be produced when the Working Electrode is held at a negative potential and then the CO is detected by sweeping the Working Electrode to positive potential. The detection of CO formation means that CO₂ is present.

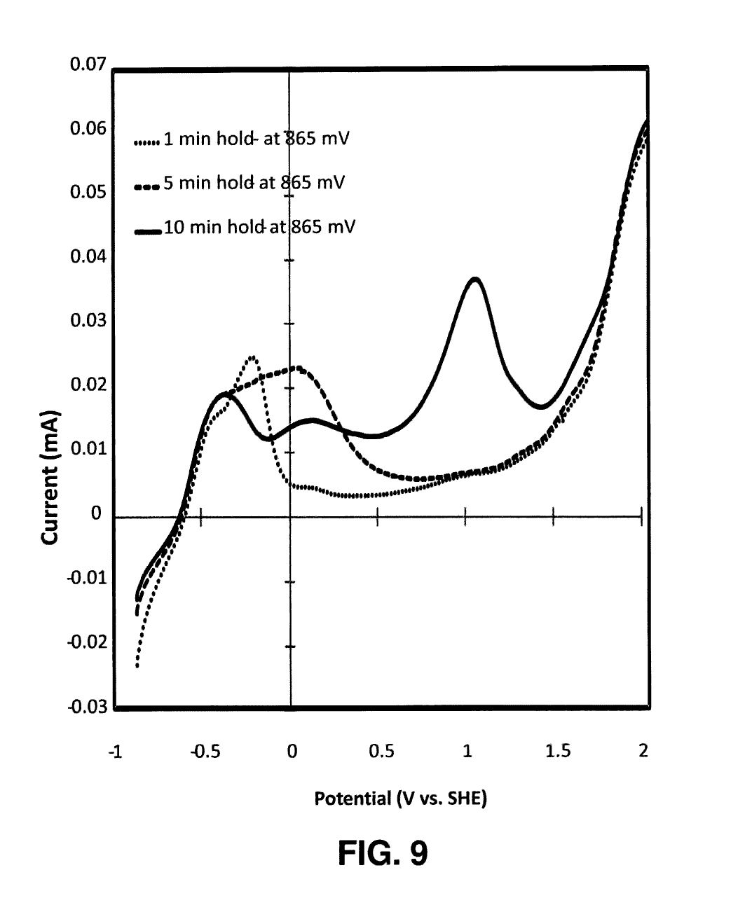

The apparatus and catalyst layer was the same as in Example 1. In this case the potential was held at −0.6 V with respect to SHE for 1, 5 and 10 minutes, and then the potential was increased at 5 mV/sec and the current was recorded. FIG. 9 shows the result. Notice the peak at about 1 V. This peak can be used to detect the presence of CO₂. This example provides an alternate way to detect CO₂ with an electrochemical sensor.

### EXAMPLE 3

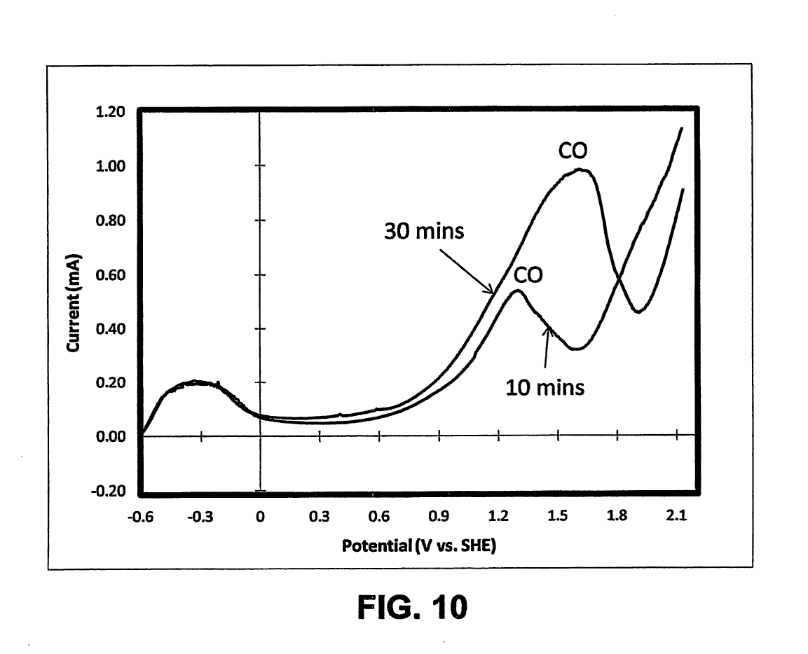

This example illustrates the effect of dilution on CO₂ sensing and shows that water additions enhance the sensitivity of the sensor. The experiment used the apparatus and procedures in Example 2, with the following exception: a solution containing 98.55% EMIM-BF4 and 0.45% water was substituted for the 99.9999% EMIM-BF4 used in Example 2, the potential was held for 10 or 30 minutes at −0.6 V with respect to RHE, and then the potential was ramped positively at 50 mV/sec. FIG. 10 shows the result. Notice the peak between 1.2 and 1.5 V. This is the peak associated with CO formation and is much larger than in Example 2. Thus the addition of water has increased the sensitivity of the sensor presumably by acting as a reactant.

### EXAMPLE 4

A CO₂ sensor with a Working Electrode including an Active Element, Helper Catalyst Mixture including silver and 1-ethyl-3-methylimidazolium tetrafluoroborate (EMIM-BF4).

This example shows that the device works with silver rather than platinum and that lower Helper Catalyst concentrations are useful.

The experiments were as in Example 1 with the following exceptions: an 18% percent EMIM-BF4 solution was substituted for the 99.9999% EMIM/BF4 in Example 1, and the Working Electrode was prepared by using 10 mg of 5 m²/gm silver nanoparticles (Sigma Aldrich) that was sonicated into a solution containing 100 μL of water, 100 μL of isopropyl alcohol and 5.6 μL of 5% Nafion® (perfluorosulfonic acid) solution (Ion Power). The resultant catalyst ink was painted on a 1×1.5 cm section of carbon paper (Ion Power) and dried with a heat lamp. The carbon paper was then used as the Working Electrode in the cell, with approximately 50% of the carbon paper immersed in the EMIM-BF4 solution.

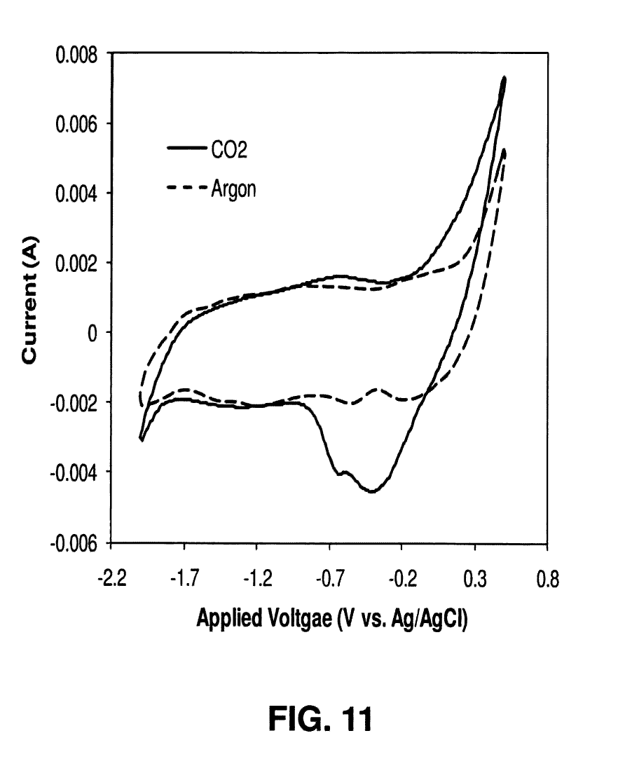

FIG. 11 compares the CV taken when argon was bubbled through the mixture to the CV when CO₂ was bubbled through the solution. There is a large negative peak near −0.5 volts with respect to silver/silver chloride associated with conversion of CO₂. The presence of this peak can be used to sense the presence of CO₂.

### EXAMPLE 5

This example demonstrates an alternate operation mode for a CO₂ sensor where CO₂ is first converted to another substance, in this example CO, in the electrochemical cell, and then the CO is detected by sweeping the working potential to positive potential to determine how much CO was produced. The presence of CO from CO₂ electrolysis can be used to detect CO₂.

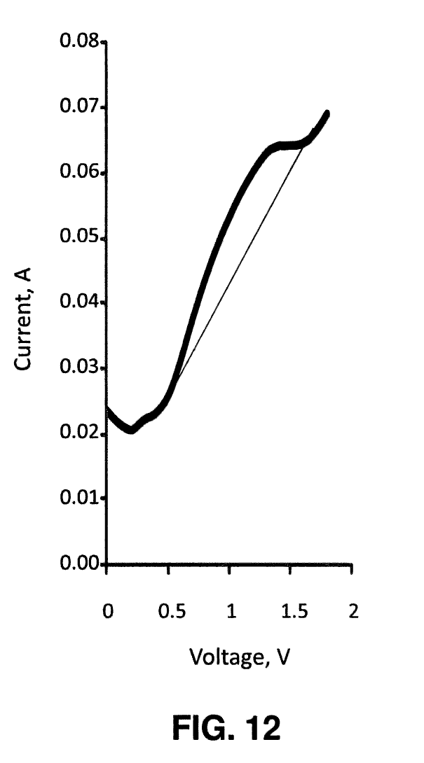

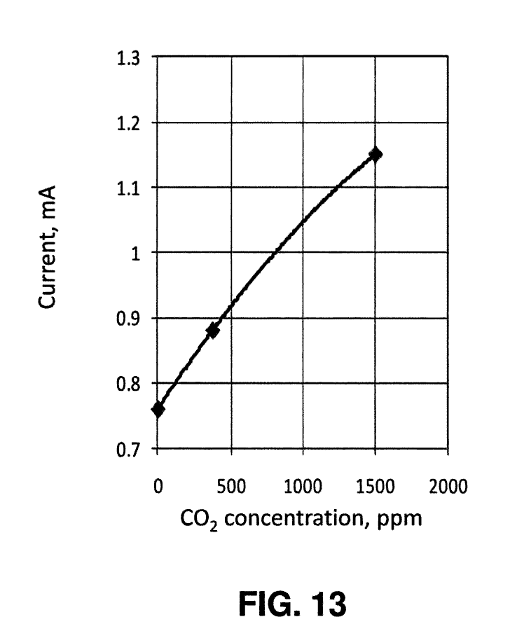

The apparatus and catalyst layer was the same as in Example 4. In this case the potential was held at −0.8 V with respect to SHE for 20 minutes, and then the potential was increased by starting at 0 V with respect to SHE, scanning at 5 mV/sec and the current was recorded. FIG. 12 shows the result. Notice the broad shoulder between 0.5 and 1 V. This shoulder can be used to measure the CO₂ concentration. This example provides an alternate way to run the electrochemical CO₂ sensor.

### EXAMPLE 6

This example, which involves the use of two different electrodes in a sensor, illustrates the concept that there can be advantages to running the device with two different electrodes: one electrode to convert the CO₂ to another substance and a second electrode to detect that substance.

The experiment used the Cell and procedures in Example 2 with the following exceptions: there were two Working Electrodes, a platinum electrode that was prepared as described in Example 1 and a silver Working Electrode prepared as in Example 4. Argon, air containing 350 ppm of CO₂ and air containing 1500 ppm of CO₂ was bubbled through the electrolyte for 20 minutes while the silver electrode was held at −3.0 V with respect to the Counter Electrode and the platinum electrode was disconnected. Then the platinum electrode was swept from −0.1 to +0.3 V with respect to a silver wire and the current was recorded on the potentiostat.

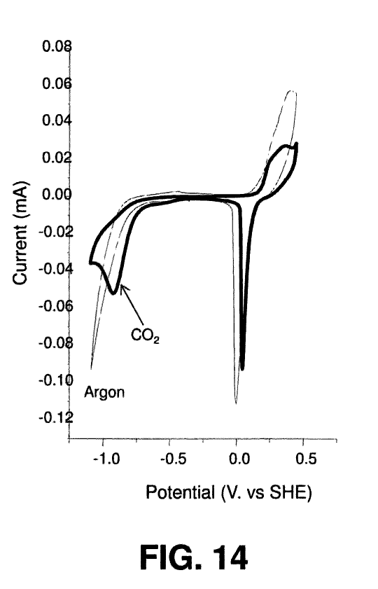

FIG. 14 shows how the current at 0.17 V varied with the CO₂ concentration. Notice that the current varies linearly with the CO₂ concentration. Clearly, this design can be used to detect CO₂.

### EXAMPLE 7

This example involves a CO₂ sensor with a Working Electrode including an Active Element, Helper Catalyst Mixture including palladium and choline iodide. It demonstrates that the present design can be practiced using palladium as an active element and choline iodide as a Helper Catalyst.

The experiment used the Cell and procedures in Example 1 with the following exceptions: (i) a 10.3% by weight of a Helper Catalyst, choline iodide, in water solution was substituted for the 1-ethyl-3-methylimidazolium tetrafluoroborate, and (ii) a 0.25 cm2 Pd foil purchased from Alfa Aesar of Ward Hill, Mass., USA, was substituted for the gold plug and platinum black on the cathode, and a silver/silver chloride reference was used.

The cell contained 52 mg of palladium and 103 mg of Helper Catalyst, so the overall catalyst mixture contained 66% of Helper Catalyst.

FIG. 14 shows a CV taken under these conditions. There is a large negative peak near zero volts with respect to SHE associated with iodine transformations and a negative going peak at about 0.8 V associated with conversion of CO₂. The height of this peak can be used to measure the CO₂ concentration.

This example also demonstrates that the invention can be practiced with a third Catalytically Active Element, palladium, and a second Helper Catalyst, choline iodide. Further, those trained in the state of the art will note that there is nothing special about the choice of palladium and choline iodide. Rather, this example shows that the results are general and not limited to the special cases in Examples 1-4.

### EXAMPLE 8

Using an Active Element, Helper Catalyst Mixture that includes palladium and choline chloride as a CO₂ sensor.

This example demonstrates that the invention can be practiced using a third Helper Catalyst, choline chloride, and that choline chloride can also act as a Hydrogen Suppressor.

The experiment used the cell and procedures in Example 7, with the following exception: a 6.5% by weight choline chloride in water solution was substituted for the choline iodide solution. The cell contained 52 mg of palladium and 65 mg of Helper Catalyst, so the overall catalyst mixture contained 51% of Helper Catalyst.

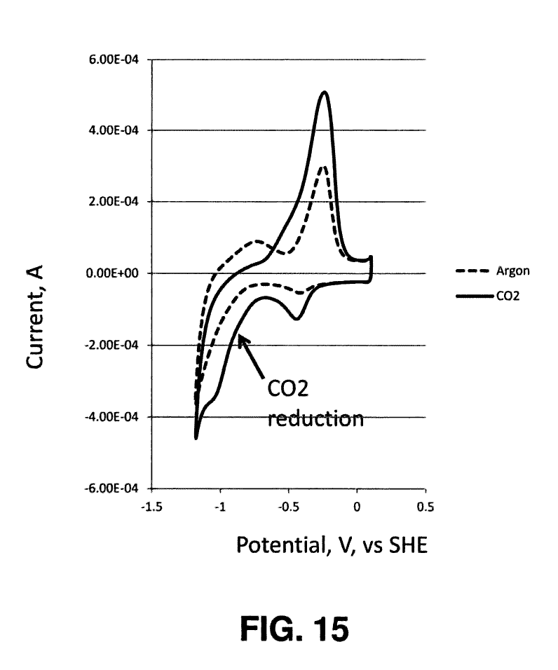

FIG. 15 shows a comparison of the cyclic voltametry for (i) a blank scan where the water-choline chloride mixture was sparged with argon and (ii) a scan where the water-choline chloride mixture was sparged with CO₂. Notice the negative going peaks starting at about −0.6. This peak can be used to detect CO₂.

Another important point is that there is no strong peak for hydrogen formation. A bare palladium catalyst would produce a large hydrogen peak at about −0.4 V at a pH of 7, while the hydrogen peak moves to −1.2 V in the presence of the choline chloride. Thus choline chloride is a Hydrogen Suppressor.

This example also demonstrates that the invention can be practiced with a third Helper Catalyst, choline chloride. Further, those trained in the state of the art will note that there is nothing special about the choice of palladium and choline chloride. Rather, this example shows that the results are general and not limited to the special case in Example 1.

### EXAMPLE 9

This example demonstrates an alternate operation mode for a CO₂ sensor where CO₂ is first converted to another substance, in this example CO, in the electrochemical cell, and then the CO is detected by sweeping the working potential to positive potential to determine how much CO was produced. The presence of CO from CO₂ electrolysis can be used to detect CO₂.

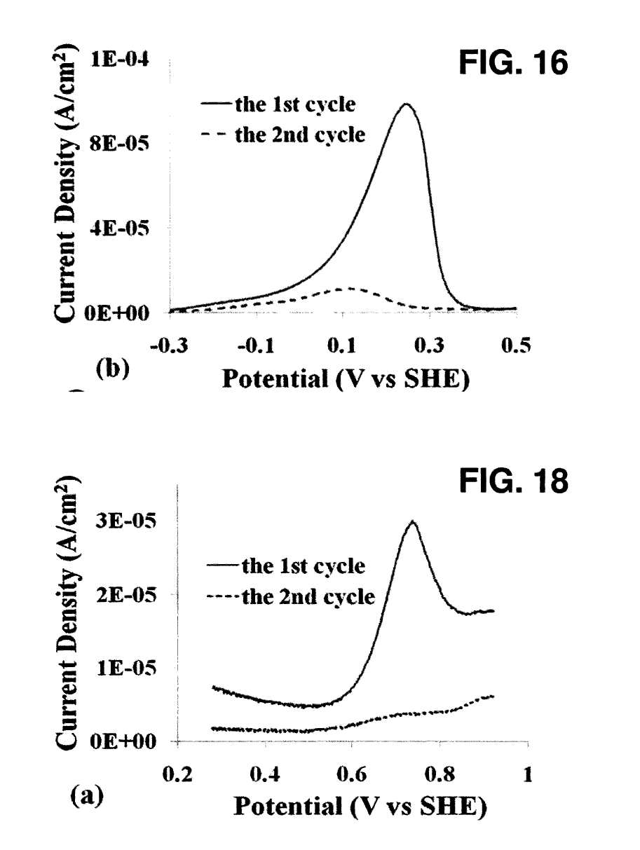

The apparatus and catalyst layer was the same as in Example 8. In this case the potential was held at −1.09 V with respect to a SHE for 10 minutes, and then the potential was increased by starting at 0 V with respect to SHE at 5 mV/sec and the current was recorded. FIG. 16 shows the result. Notice the broad shoulder between 0.5 and 1 V. This shoulder can be used to measure the CO₂ concentration. This example provides an alternate way to operate the electrochemical CO₂ sensor.

### EXAMPLE 10

Using an Active Element, Helper Catalyst Mixture that includes nickel and choline chloride as a CO₂ sensor.

The next example is to demonstrate that the invention can be practiced using a fourth metal, nickel.

The experiment used the Cell and procedures in Example 8, with the following exception: a nickel foil from Alfa Aesar was substituted for the palladium foil.

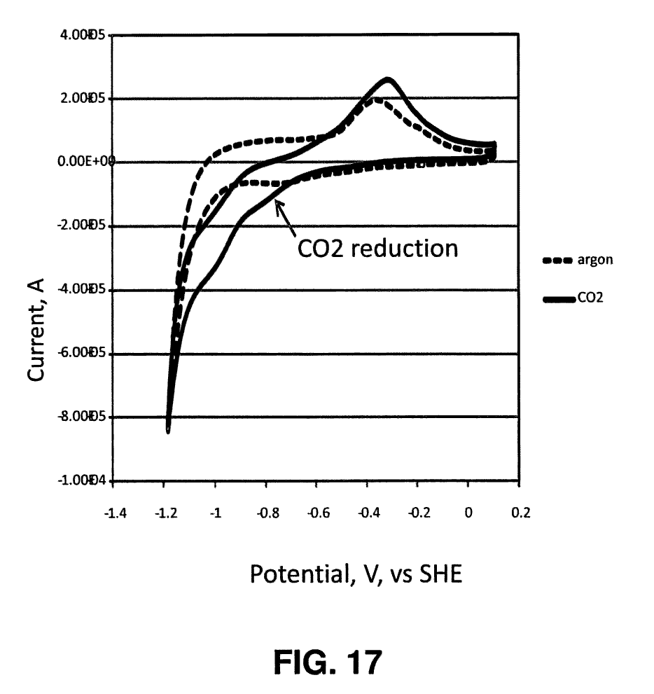

FIG. 17 shows a comparison of the cyclic voltametry for (i) a blank scan where the water-choline chloride mixture was sparged with argon and (ii) a scan where the water-choline chloride mixture was sparged with CO₂. Notice the negative going peaks starting at about −0.6. The present result shows that CO₂ is being reduced at −0.6 V. A voltage more negative than −1.48 V is required to convert CO₂ on nickel in the absence of the Helper Catalyst. Thus, the Helper Catalyst has lowered the overpotential for CO₂ conversion.

Another important point is that there is no strong peak for hydrogen formation. A bare nickel catalyst would produce a large hydrogen peak at about −0.4 V at a pH of 7, while the hydrogen peak moves to −1.2 V in the presence of the Helper Catalyst. The Hori Review reports that nickel is not an effective catalyst for CO₂ reduction because the side reaction producing hydrogen is too large. The data in FIG. 17 show that the Helper Catalysts are effective in suppressing hydrogen formation.

### EXAMPLE 11

This example also demonstrates that the present technique can be practiced with a third metal, nickel. Further, those trained in the state of the art will note that there is nothing special about the choice of nickel and choline chloride. Rather, this example shows that the results are general and not limited to the special case in Example 1. FIG. 18 Shows a shows a CO stripping experiment done by starting with a catalyst mixture where the catalyst was synthesized as in Example 10 saturating the choline chloride solution with CO₂, holding the potential at −0.6 V with respect to SHE for 20 minutes and then ramping the potential from −0.6 to +2V and measuring the current.

Those familiar with the technology involved here should realize that since choline chloride and choline iodide are active, other choline salts such as choline bromide, choline fluoride and choline acetate should be active as well.

### EXAMPLE 12

In the paragraphs above in which the two-cell embodiment is first described, it is noted that the CO₂ sensor can be a single sensor designed as in FIG. 1, with a membrane electrode assembly (MEA) that includes a Working Electrode 110, an ion conducting polymer membrane 111 and a Counter Electrode 112. The MEA is sandwiched between two hydrophobic current collectors 113 and 114. The device sits in a housing 115 with a water reservoir 116. The design can also be a dual sensor design as in FIG. 2 with two similar designs and possibly different catalysts. The objective of this Example 12 is to make explicit what was implicit in the earlier description of the two-cell embodiment.

First, a membrane was formed by starting with a 4 cm×6 cm porous PTFE structure (available under the trade designation Porex PM15M, Porex Corporation, Fairburn, Ga., USA). PSMMIM was prepared as described in U.S. Pat. App. Pub. No. US2016/0107154A1 (published version of co-owned U.S. patent application Ser. No. 14/704,935), where “PSMMIM” refers to a co-polymer of polystyrene and poly 1-(p-vinylbenzyl)-3-methyl-imidazolium:

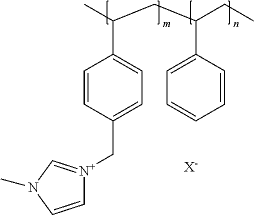

where X⁻ is an anion, m>0 and n>0.
3 ml of 20% PSMMIM by weight in ethanol was painted onto the porous structure. The resultant structure was heated in an oven to 80° C. for 1 hour and another 30 minutes at 120° C. to create an ion conducting membrane.

Next, a cathode ink was prepared as follows. First, a 5% by weight ethyl cellulose solution was prepared by dissolving ethyl cellulose (Sigma Aldrich, 200689, viscosity 10 cP) in ethanol (Sigma Aldrich). Next, 0.2 grams of silver nanoparticles (20-40 nm, 45509, Alfa Aesar, Ward Hill, Mass.), was mixed with 10 mg porous carbon (Vulcan XC-72R, Fuel Cell Earth, Woburn, Mass.), 1 ml of isopropanol and 1 ml of the ethyl cellulose solution. The mixture was then sonicated for 10 minutes.

Next, the cathode ink was painted onto the ion conducting membrane. The resultant structure was dried at 80° C. for 5 minutes.

Similarly, an anode ink was created by mixing 0.2 g of ruthenium oxide particles, in 2 ml of 1% Teflon® AF solution (DuPont). The anode ink was painted onto the ion conducting membrane. The resultant structure was dried at 80° C. for 5 minutes to create an MEA. The as-prepared MEA sensor was soaked in 1 M KOH for 24 hours before use.

Next, a 7 mm circular pattern was cut out of the MEA and assembled in a Plexiglass® polycarbonate housing with water in the bottom as indicated in FIG. 1. Titanium ribbons were used to make contact with the anode and cathode in the MEA. The resultant sensor showed an approximately 50 μA change in current when the CO₂ concentration was changed from 0 ppm to 400 ppm CO₂.

We also created a double cell structure. In this case two identical MEA's prepared as above were mounted in a Plexiglas housing. One of the sensors was directly exposed to the environment, while the second was covered with a Nafion® 221 (DuPont) perfluorosulfonic acid film so the second sensor could act as a reference. Again we saw an approximately 50 μA change in current when the CO₂ concentration was changed from 0 ppm to 400 ppm CO₂ on the sensor that was directly exposed to the environment and only about a 1 μA change in current in the sensor that was covered with Nafion® perfluorosulfonic acid film. This design can compensate for temperature variations in the environment.

A similar design using PSMP in place of PSMMIM was also tested, where “PSMP” refers to a co-polymer of polystyrene and poly 1-(p-vinylbenzyl)-pyridinium. The PSMP was manufactured as described in U.S. Pat. App. Pub. No. US2016/0107154A1. 3 ml of 6% PSMP by weight in ethanol was painted onto the porous structure. The resultant structure was heated in an oven to 80° C. for 1 hour and another 30 minutes at 120° C. to produce an ion conducting membrane. A sensor was then fabricated as described above in this example for the as-prepared MEA sensor.

The resultant sensor showed an approximately 80 nA change in current when the CO₂ concentration was changed from 0 ppm to 400 ppm CO₂.

A design employing PSPZ in place of PSMMIM was also tested, where “PSPZ” refers to a co-polymer of polystyrene and poly 1-(p-vinylbenzyl)-pyrazolium. First, a copolymer of styrene and 1-(p-vinylbenzyl chloride was prepared by anionic polymerization as described in U.S. Pat. App. Pub. No. US2016/0107154A1. Pyrazole (Sigma-Aldrich) (0.593 g, 4.67 mmol) was added to the solution containing 1 g (3.89 mmol) of the styrene vinylbenzyl chloride copolymer in anhydrous N,N-Dimethylformamide (DMF) (Sigma-Aldrich) (8 mL). The mixture was stirred at room temperature for 60 hours to create a mixture of PSPZ in DMF.

A 6% PSPZ in DMF mixture was painted onto the porous structure. The resultant structure was heated in an oven to 80° C. for 1 hour and another 30 minutes at 120° C. to create an ion conducting membrane. A sensor was then fabricated as described above in this example for the as-prepared MEA sensor.

The resultant sensor showed an approximately 50 nA change in current when the CO₂ concentration was changed from 0 ppm to 400 ppm CO₂.

Finally, an experiment was conducted to examine the effect of adding a Hydrogen Suppressor described in the earlier paragraphs regarding Hydrogen Suppressors. In particular, 1 ml of an 18% by weight choline bicarbonate was added to the cathode ink before painting. This had the effect of reducing the effect of humidity, when the water reservoir was dry.

These examples demonstrate that the sensor design in the earlier description of the two-cell embodiment performs advantageously, and that use of the hydrogen suppressors can be advantageous. The results also show that membranes containing many different positively charged cyclic amines can be employed to detect CO₂.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in the chemical arts or in the relevant fields are intended to be within the scope of the appended claims.

The disclosures of all references and publications cited above are each expressly incorporated by reference in its entirety to the same extent as if each were incorporated by reference individually.

What is claimed is:

1. A carbon dioxide sensor comprising an electrochemical cell comprising at least one Working Electrode, at least one Counter Electrode, a polymer electrolyte, and a Catalytically Active Element, wherein the electrochemical cell is active for the electrochemical reduction of CO₂.

2. The carbon dioxide sensor of claim 1, wherein the polymer electrolyte comprises a charged cyclic amine.

3. The carbon dioxide sensor of claim 2, wherein the polymer electrolyte comprises at least one of an imidazolium, a pyridinium and a pyrazolium.

4. The carbon dioxide sensor of claim 1, wherein the polymer electrolyte comprises polymerized styrene monomers.

5. The carbon dioxide sensor of claim 3, wherein at least one Working Electrode is active for electrochemical reduction of CO₂ into another substance S.

6. The carbon dioxide sensor of claim 5, wherein the substance S comprises at least one constituent selected from the group consisting of CO, HCO⁻, H₂CO, (HCO₂)⁻, H₂CO₂, CH₃OH, CH₄, C₂H₄, CH₃CH₂OH, CH₃COO⁻, CH₃COOH, C₂H₆, O₂, (COOH)₂, and (COO⁻)₂.

7. The carbon dioxide sensor of claim 2, wherein the Catalytically Active Element comprises at least one constituent selected from the group consisting of V, Cr, Mn, Fe, Co, Ni, Cu, Sn, Zr, Nb, Mo, Ru, Rh, Pd, Ag, Cd, Hf, Ta, W, Re, Ir, Pt, Au, Hg, Al, Si, In, Tl, Pb, Bi, Sb, Te, U, Sm, Tb, La, Ce and Nd.

8. The carbon dioxide sensor of claim 2, wherein said Catalytically Active Element is selected from the group consisting of Ru, Ir, Ni, Pt, Pd, Ag, Cu, Au and Sn.

9. The carbon dioxide sensor of claim 1, further comprising at least one of (i) a Helper catalyst, (ii) a Director Substance, and (iii) Hydrogen Suppressor.

10. The carbon dioxide sensor of claim 9, wherein the Hydrogen Suppressor comprises at least one constituent selected from the group consisting of choline chlorine, tetrabutylammonium hydrogen sulfate (TBAHS) and ethylenediaminetetraacetic acid (EDTA), benzaldehyde and substituted benzaldehydes, di-acids such as succinic acid and substituted di-acids, an ionic liquid, and a compound of the form R₁R₂R₃N⁺(CH₂)ₙOH, R₁R₂R₃N⁺(CH₂)ₙCOH or R₁R₂R₃N⁺(CH₂)ₙCOOH wherein n=1-4 and R₁, R₂ and R₃ are each a ligand containing at least 1 carbon atom.

11. The carbon dioxide sensor of claim 10, wherein said Hydrogen Suppressor comprises a choline cation or a choline derivative of the form R₁R₂R₃N⁺(CH₂)ₙOH, R₁R₂R₃N⁺(CH₂)ₙCOH or R₁R₂R₃N⁺(CH₂)ₙCOOH, wherein n=1-4, and R₁, R₂ and R₃ are each a ligand containing at least 1 carbon atom.

12. The carbon dioxide sensor of claim 10, wherein, R₁, R₂ and R₃ are independently selected from the group consisting of aliphatic C₁-C₄ groups, —CH₂OH, —CH₂CH₂OH, —CH₂CH₂CH₂OH, —CH₂CHOHCH₃, —CH₂COH, —CH₂CH₂COH, and —CH₂COCH₃ and molecules, wherein one of more chlorine or fluorine is substituted for the hydrogens in aliphatic C₁-C₄ groups, —CH₂OH, —CH₂CH₂OH, —CH₂CH₂CH₂OH, —CH₂CHOHCH₃, —CH₂COH, —CH₂CH₂COH, and —CH₂COCH₃.

13. The carbon dioxide sensor of claim 9, wherein the Director Substance comprises at least one constituent selected from the group consisting of ionic liquids, deep eutectic solvents, amines, and phosphines.

14. The carbon dioxide sensor of claim 9, wherein the Director Substance comprises at least one constituent selected from the group consisting of imidazoliums, pyridiniums, pyrrolidiniums, phosphoniums, ammoniums, sulfoniums, prolinates, methioninates, acetylcholines, alanines, aminoacetonitriles, methylammoniums, arginines, aspartic acids, cholines, threonines, chloroformamidiniums, thiouroniums, quinoliniums, pyrrolidinols, serinols, benzamidines, sulfamates, acetates, carbamates, triflates, cyanides, amines, phosphonates, polyimides, and other nitrogen-containing or phosphorous-containing polymers and anion exchange resins.

15. An electrochemical system, the system comprising a carbon dioxide sensor comprising at least one Working Electrode, at least one Counter Electrode, a polymer electrolyte, and a Catalytically Active Element, wherein the Working Electrode is active for the electrochemical reduction of CO₂.

16. The carbon dioxide sensor of claim 15, wherein the polymer electrolyte comprises a charged cyclic amine.

17. The carbon dioxide sensor of claim 15, wherein the polymer electrolyte comprises polymerized styrene monomers.

18. The carbon dioxide sensor of claim 15, wherein the polymer electrolyte comprises at least one of an imidazolium, a pyridinium and a pyrazolium.

19. The system of claim 15, wherein the sensor further comprises a CO₂ permeable polymer.

20. The system of claim 15, wherein the system is capable of monitoring dissolved CO₂ levels in bodily fluids comprising at least one constituent selected from the group consisting of blood serum, bile, gastric fluid, saliva and urine, the system thereby being capable of performing medical diagnostics.

21. The system of claim 15, wherein the system is capable of monitoring dissolved CO₂ levels in an environment selected from the group consisting of glacial, ocean, runoff, rain, aquifer, river, estuary, and pond water.

22. The system of claim 15, wherein the system is capable of monitoring CO₂ levels in liquid phase reactors, fermenters, industrial liquid streams, and industrial byproducts.
